(19) United States
(12) Patent Application Publication
(10) Pub. No.: US 2023/0017150 A1
(43) Pub. Date: Jan. 19, 2023

(54) HYDROGEL STENT AND EMBOLIZATION DEVICE FOR CEREBRAL ANEURYSM

(71) Applicants: THE ASAN FOUNDATION, Seoul (KR); UNIVERSITY OF ULSAN FOUNDATION FOR INDUSTRY COOPERATION, Ulsan (KR)

(72) Inventors: Deok Hee LEE, Seoul (KR); Seon Moon HWANG, Gyeonggi-do (KR); Joon Ho CHOI, Seoul (KR)

(73) Assignees: THE ASAN FOUNDATION, Seoul (KR); UNIVERSITY OF ULSAN FOUNDATION FOR INDUSTRY COOPERATION, Ulsan (KR)

(21) Appl. No.: 17/812,892

(22) Filed: Jul. 15, 2022

(30) Foreign Application Priority Data
Jul. 16, 2021 (KR) 10-2021-0093795

(51) Int. Cl. A61B 17/12

(52) U.S. Cl. CPC A61B 17/12113; A61B 2017/1205; A61B 2090/3966

(57) ABSTRACT

A hydrogel stent for cerebral aneurysm includes a hydrogel polymer, and a stent body wrapping at least a portion of the hydrogel polymer. The stent body is expandable in a cerebral aneurysm of a blood vessel. The stent body is provided in a mesh type surrounding at least a portion of the hydrogel polymers. An embolization device for cerebral aneurysm, includes a hydrogel stent for cerebral aneurysm; a micro-conduit providing a movement path for guiding the hydrogel stent to a cerebral aneurysm of a blood vessel; and a pusher wire connected to the hydrogel stent to be movable in the movement path.

### TECHNICAL FIELD

The present disclosure relates to a hydrogel stent and an embolization device for a cerebral aneurysm.

### BACKGROUND

In general, a cerebral aneurysm is a vascular disease in which a cerebrovascular barrier is abnormally bulged and a microscopic crack is generated in the cerebrovascular barrier.

A cerebral aneurysm may irritate the meninges and cause vomiting, nausea, a severe headache, and a stiff neck, and in severe cases of cerebral aneurysms, the pressure within the skull increases, which causes a person to lose consciousness and fall into a coma, which may lead to death, or leave very serious sequelae.

To treat such cerebral aneurysms, cerebral aneurysm ligation and cerebral aneurysm coil embolization can be applied. Among them, cerebral aneurysm coil embolization involves inserting a micro conduit into the cerebral aneurysm and filling the cerebral aneurysm with a coil of an embolic material (e.g., platinum, etc.) to prevent a hematocele from entering the cerebral aneurysm.

However, in the case of conventional cerebral aneurysm coil embolization, the coil of the embolic material may be separated from the cerebral aneurysm depending on the size of a diameter of the cerebral aneurysm or the shape of the cerebral aneurysm.

(Prior Patent Document 1) Korean Patent Publication No. 10-2016-0022597 (published on Mar. 2, 2016)

### SUMMARY

The present disclosure embodiments provide a hydrogel stent and an embolization device for a cerebral aneurysm capable of embolizing a cerebral aneurysm without separation of the hydrogel polymer by inserting a hydrogel polymer into the cerebral aneurysm.

In accordance with a first aspect of the present disclosure, there is provided a hydrogel stent for cerebral aneurysm, including: a hydrogel polymer; and a stent body wrapping at least a portion of the hydrogel polymer, the stent body being expandable in a cerebral aneurysm of a blood vessel.

The stent body may be provided in a mesh type surrounding at least a portion of the hydrogel polymers.

The hydrogel polymer may be entirely accommodated in an inner space of the stent body, or a portion of the hydrogel polymer may be accommodated in the stent body to be exposed outside.

The stent body may be formed of a Nitinol material that is restored to its original shape when its temperature is higher than a predetermined temperature or when it is in contact with water.

The stent body may include: a web portion in which a plurality of stent wires is convexly connected to each other so as to provide an inner space in which the hydrogel polymer is accommodated; a converging portion provided at one end of the web portion so that a distal side of the web portion converges; and a separation portion provided at the other end of the web unit so that a proximal side of the web portion converges, the separation portion being connected to a pusher wire, and detachable when the stent body is inserted into the cerebral aneurysm.

The hydrogel stent may further include a filament for supporting the stent body, wherein the filament includes: a filament body having a predetermined rigidity for supporting the stent body; first coupling parts configured to be respectively coupled the stent body and provided at longitudinal ends of the filament body; and a second coupling part configured to be coupled to a pusher wire and provided at one of the longitudinal ends of the filament body, wherein the filament body extends from one of longitudinal ends of the stent body to the other of the longitudinal ends of the stent body viewed in a longitudinal direction of the stent body so that a diameter in the longitudinal direction of the hydrogel stent unfolded is maintained substantially as same as that of the hydrogel stent folded.

The filament may be formed by coating or attaching the hydrogel polymer to a surface of a metal material

In accordance with a second aspect of the present disclosure, there is provided an embolization device for cerebral aneurysm, including: a hydrogel stent for cerebral aneurysm; a micro-conduit providing a movement path for guiding the hydrogel stent to a cerebral aneurysm of a blood vessel; and a pusher wire connected to the hydrogel stent to be movable in the movement path; wherein the hydrogel stent includes: a hydrogel polymer; and a stent body wrapping at least a portion of the hydrogel polymer, the stent body being expandable in the cerebral aneurysm of the blood vessel.

The embolization device may further include: a radiopaque marker provided to the hydrogel stent and made of a material that does not pass radiation to confirm a location of the hydrogel stent in the blood vessel.

The hydrogel stent may be expanded in the cerebral aneurysm while being discharged from a tip of the micro-conduit by being pushed by the pusher wire as the tip of the micro-conduit is inserted into the cerebral aneurysm.

The hydrogel stent may be provided in a state of being inserted in the micro-conduit before a surgical procedure for the cerebral aneurysm, and the stent body may be maintained in a state in which an outer surface thereof is compressed by an inner surface of the micro-conduit.

According to the embodiments of the present disclosure, it is possible to perform an embolization without detachment of the hydrogel polymer in the cerebral aneurysm by inserting a hydrogel polymer which absorbs blood and swells into the cerebral aneurysm.

### BRIEF DESCRIPTION OF THE DRAWINGS

FIG. 1 is a block diagram illustrating an embolization device for a cerebral aneurysm according to a first embodiment of the present disclosure.

FIG. 2 is a state diagram illustrating a state in which the embolization device is inserted into the cerebral aneurysm according to the first embodiment of the present disclosure.

FIG. 3 is a state diagram illustrating a state in which a stent body of the embolization device is unfolded in the cerebral aneurysm according to the first embodiment of the present disclosure.

FIG. 4 is a state diagram illustrating a state in which the pusher wire is separated from the stent body of the embolization device according to the first embodiment of the present disclosure.

FIG. 5 is a front view showing a state in which a hydrogel stent for cerebral aneurysm is inserted into the cerebral aneurysm according to a second embodiment of the present disclosure.

FIG. 6 is a side view showing the hydrogel stent for cerebral aneurysm according to the second embodiment of the present disclosure.

FIG. 7 is a front view illustrating a state in which a hydrogel stent for cerebral aneurysm is inserted into the cerebral aneurysm according to a modified example of the second embodiment of the present disclosure.

FIG. 8 is a side view illustrating a hydrogel stent for cerebral aneurysm according to the modified example of the second embodiment of the present disclosure.

FIG. 9 is a front view illustrating a state in which a hydrogel stent for cerebral aneurysm is inserted into the cerebral aneurysm according to another modification of the second embodiment of the present disclosure.

FIG. 10 is a side view illustrating a hydrogel stent for cerebral aneurysm according to another modification of the second embodiment of the present disclosure.

FIG. 11 is a front view illustrating a state in which a hydrogel stent for cerebral aneurysm is inserted into a cerebral aneurysm according to a third embodiment of the present disclosure.

FIG. 12 is a side view showing a hydrogel stent for cerebral aneurysm according to the third embodiment of the present disclosure.

FIG. 13 is a front view illustrating a state in which a hydrogel stent for cerebral aneurysm is inserted into a cerebral aneurysm according to a modified example of the third embodiment of the present disclosure.

FIG. 14 shows a hydrogel stent for cerebral aneurysm according to a fourth embodiment of the present disclosure, and FIG. 15 illustrates a state in which a hydrogel stent for cerebral aneurysm is inserted into a cerebral aneurysm according to the fourth embodiment of the present disclosure.

FIG. 16 shows a hydrogel stent for cerebral aneurysm which includes first coupling parts having a hook shape, and FIG. 17 illustrates a state in which the hydrogel stent for cerebral aneurysm shown in FIG. 16 is inserted into a cerebral aneurysm.

### DETAILED DESCRIPTION

Hereinafter, specific embodiments for implementing a spirit of the present disclosure will be described in detail with reference to the drawings.

In describing the present disclosure, detailed descriptions of known configurations or functions may be omitted to clarify the present disclosure.

When an element is referred to as being ‘connected’ to, ‘supported’ by, ‘accessed’ to, ‘supplied’ to, ‘transferred’ to, or ‘contacted’ with another element, it should be understood that the element may be directly connected to, supported by, accessed to, supplied to, transferred to, or contacted with another element, but that other elements may exist in the middle.

The terms used in the present disclosure are only used for describing specific embodiments, and are not intended to limit the present disclosure. Singular expressions include plural expressions unless the context clearly indicates otherwise.

Further, in the present disclosure, it is to be noted that expressions, such as the upper side and the lower side, are described based on the illustration of drawings, but may be modified if directions of corresponding objects are changed. For the same reasons, some components are exaggerated, omitted, or schematically illustrated in the accompanying drawings, and the size of each component does not fully reflect the actual size.

Terms including ordinal numbers, such as first and second, may be used for describing various elements, but the corresponding elements are not limited by these terms. These terms are only used for the purpose of distinguishing one element from another element.

In the present specification, it is to be understood that the terms such as “including” are intended to indicate the existence of the certain features, areas, integers, steps, actions, elements, combinations, and/or groups thereof disclosed in the specification, and are not intended to preclude the possibility that one or more other certain features, areas, integers, steps, actions, elements, combinations, and/or groups thereof may exist or may be added.

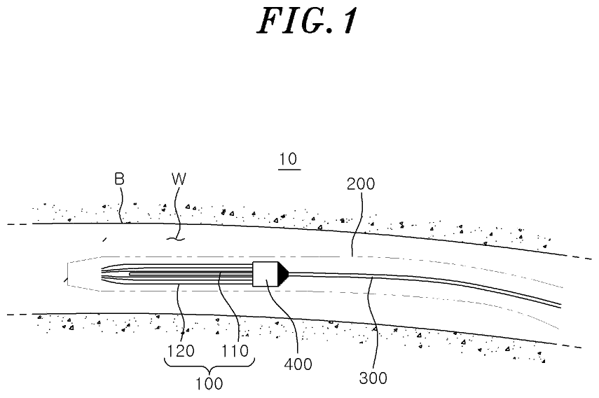

Hereinafter, a detailed configuration of the cerebral aneurysm hydrogel stent according to an embodiment of the present invention will be described with reference to FIGS. 1 to 12.

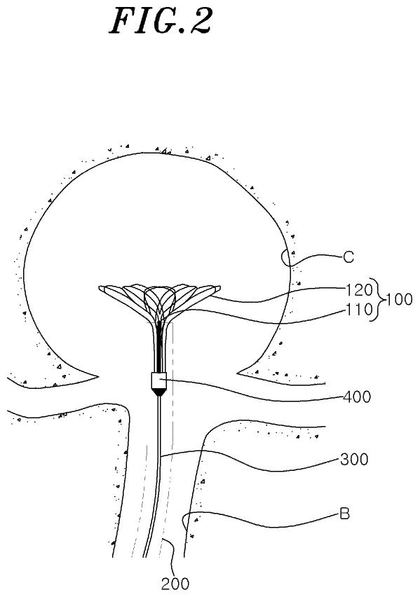

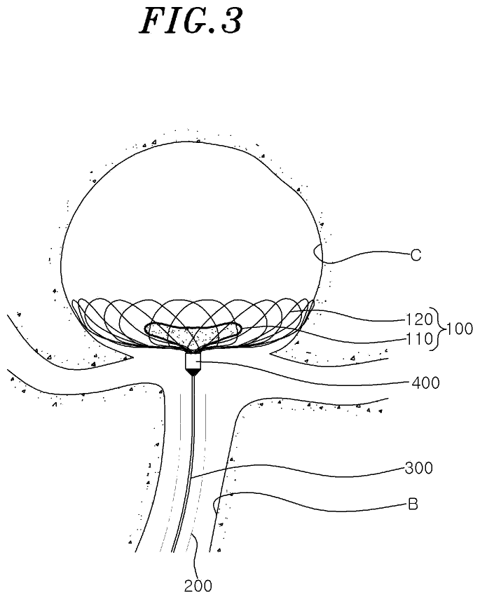

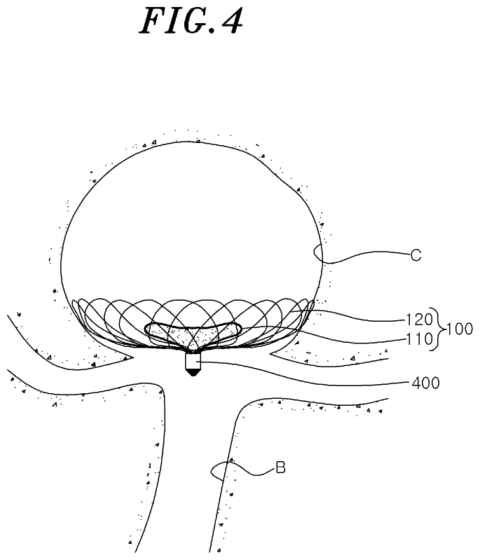

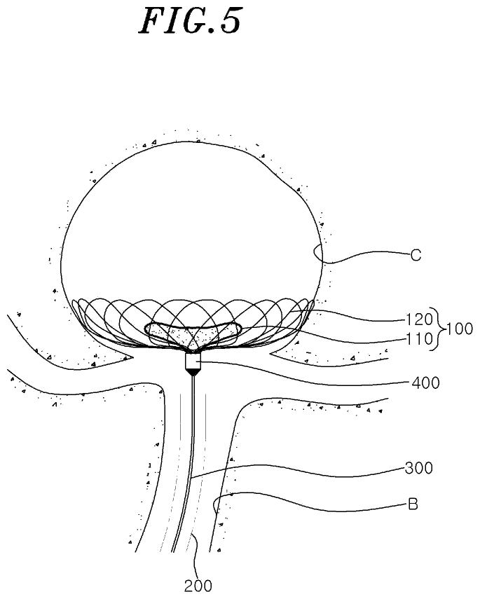

FIG. 1 is a block diagram illustrating an embolization device for a cerebral aneurysm according to a first embodiment of the present disclosure, FIG. 2 is a state diagram illustrating a state in which the embolization device is inserted into the cerebral aneurysm according to the first embodiment of the present disclosure, FIG. 3 is a state diagram illustrating a state in which the stent of the embolization device is unfolded in the cerebral aneurysm according to the first embodiment of the present disclosure, FIG. 4 is a state diagram illustrating a state in which the pusher wire is separated from the stent of the embolization device according to the first embodiment of the present disclosure.

As shown in FIGS. 1 to 4, the cerebral aneurysm embolic device 10 according to the first embodiment of the present disclosure may insert a hydrogel polymer 110 into the cerebral aneurysm C to prevent the hydrogel polymer from being separated from the cerebral aneurysm. The cerebral aneurysm embolic device 10 may include a hydrogel stent for cerebral aneurysm 100, a microconduit 200, a pusher wire 300, and a radiopaque marker 400.

Specifically, the hydrogel stent 100 may include the hydrogel polymer 110 and the stent body 120. Hydrogel polymer 110 is a superabsorbent electrolyte polymer using water as a dispersion medium, and when the hydrophilic polymer meets blood, it can absorb blood and expand. The hydrogel polymer 110 may undergo a phase transition with temperature, pH, etc., so that the expansion ratio may be discontinuously changed. The hydrogel polymer 110 may be a biodegradable hydrogel or a non-biodegradable hydrogel.

The hydrogel polymer 110 may be accommodated in the stent body 120. The diameter and length of the hydrogel polymer 110 may be adjusted according to the cell size and shape of the stent body 120.

The stent body 120 in which the hydrogel polymer 110 is accommodated may remain inserted into the microconduit 200 until it is inserted into the cerebral aneurysm C. Since the hydrogel polymer 110 is maintained in a state separated from the blood in the micro conduit 200, the hydrogel polymer 110 may maintain a state before expansion. At this time, an expansion volume of the hydrogel polymer 110 may be adjusted according to the degree of expansion of the stent body 120.

The hydrogel polymer 110 may be discharged from the micro conduit 200 and inserted into the cerebral aneurysm C together with the stent body 120 while positioned close to the cerebral aneurysm C. When the hydrogel polymer 110 is inserted into the cerebral aneurysm C, the hydrogel polymer 110 may swell by absorbing blood within the cerebral aneurysm C.

Especially, when the hydrogel polymer 110 is expanded in the cerebral aneurysm C, the hydrogel polymer 110 may expand within the cerebral aneurysm C to correspond to the shape of an inner surface of the cerebral aneurysm C. In this case, an outer surface of the hydrogel polymer 110 expanded in the cerebral aneurysm C may have a shape corresponding to the inner surface of the cerebral aneurysm C. As a result, the hydrogel polymer 110 swollen in the cerebral aneurysm C matches the internal shape of the cerebral aneurysm C and may not be separated from the cerebral aneurysm C.

In this embodiment, the hydrogel polymer 110 has been described as accommodated in the inner space of the stent body 120, but is not limited thereto, and the hydrogel polymer 110 may be integrally coupled with the stent body 120. As an example, the starting point (lower end) of the hydrogel polymer 110 and the starting point (lower end) of the stent body 120 may be coupled to each other. In addition, the hydrogel polymer 110 may be coupled to each other by twisting the stent wire of the stent body 120, or the stent wire of the stent body 120 may penetrate and be coupled to the hydrogel polymer 110.

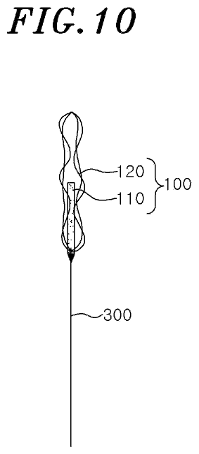

The stent body 120 may include a plurality of stent wires surrounding the hydrogel polymer 110. In the case the stent body 120 wraps around the hydrogel polymer 110 as a whole, since the flow rate of blood flowing into the cerebral aneurysm C is different depending on the cell size or shape between the stent wires of the stent body 120, the diameter and length of the hydrogel polymer 110 may be adjusted in consideration of the flow rate of blood. For example, a diameter and a length of the hydrogel polymer 110 coupled to the stent having a large cell size (see, FIGS. 4 to 9) may be greater than the diameter and length of the hydrogel polymer 110 coupled to the stent having a small cell size (see, FIGS. 10 to 12).

The stent body 120 may be provided in the form of a basket or a fishnet which surrounds at least a portion of the hydrogel polymer 110, and the stent body 120 may be made of Nitinol. The stent body 120 made of Nitinol may have a property of being restored to its original shape when it reaches a predetermined temperature or more or meets water (blood).

The stent body 120 may be maintained in a state of being inserted into the microconduit 200 until it is inserted into the cerebral aneurysm C. For example, in a state in which the stent body 120 is inserted into the micro-conduit 200 before being inserted into the cerebral aneurysm C, the compressed state may be maintained by being pressed by the inner surface of the micro-conduit 200.

When the end of the micro-conduit 200 is inserted into the cerebral aneurysm C, the stent body 120 may be unfolded while being discharged toward the cerebral aneurysm C by the push of the pusher wire 300. For example, when the stent body 120 is discharged from the micro-conduit 200 by the push of the pusher wire 300, the stent body 120 may be expanded while being restored to its original shape.

The micro conduit 200 may be provided in the form of a transparent hollow tube. The micro-conduit 200 may guide the hydrogel stent 100 to the cerebral aneurysm C of the blood vessel B. The hydrogel stent 100 may be positioned at the end portion of the micro-conduit 200 so that the hydrogel stent 100 may go into or exit from the micro-conduit 200. When the end portion of the micro-conduit 200 is inserted into the cerebral aneurysm C, the hydrogel stent 100 may be discharged from the end portion of the micro-conduit 200 and unfolded.

The pusher wire 300 may be provided in the form of a wire for guiding the hydrogel stent 100 to the cerebral aneurysm C. The pusher wire 300 may be inserted into the micro conduit 200 through the sheath tube.

The radiopaque marker 400 may be provided in the hydrogel stent 100. The radiopaque marker 400 may be made of a material that does not pass radiation. Since the radiopaque marker 400 does not pass radiation, the position of the hydrogel stent 100 in the blood vessel B can be confirmed through the radiopaque marker 400 during radiographic examination.

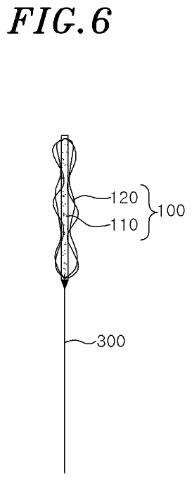

FIG. 5 is a front view showing a state in which the cerebral aneurysm hydrogel stent is inserted into the cerebral aneurysm according to a second embodiment of the present disclosure, and FIG. 6 is a side view showing a cerebral aneurysm hydrogel stent according to the second embodiment of the present disclosure.

Meanwhile, as shown in FIGS. 5 to 6, according to the second embodiment, the hydrogel polymer 110 having a volume corresponding to the inner space of the stent body 120 may be accommodated in the inner space of the stent body 120. The overall length of the hydrogel polymer 110 and the height of the inner space of the stent body 120 may be the same or similar to each other. The stent wire of the stent body 120 may be connected to each other to prevent the hydrogel polymer 110 from escaping to the outside.

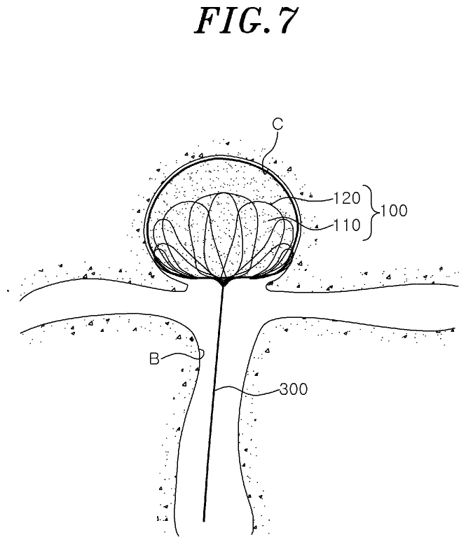

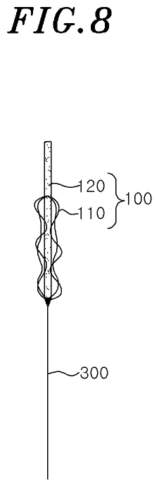

FIG. 7 is a front view illustrating a state in which a cerebral aneurysm hydrogel stent is inserted into the cerebral aneurysm according to a modified example of the second embodiment of the present disclosure, and FIG. 8 is a side view illustrating a cerebral aneurysm hydrogel stent according to a modified example of the second embodiment of the present disclosure.

As shown in FIGS. 7 and 8, according to the modified example of the second embodiment, in the inner space of the stent body 120, the hydrogel polymer 110 having a smaller volume than the volume of the inner space of the stent body 120 may be accommodated. The overall length of the hydrogel polymer 110 may be shorter than the height of the inner space of the stent body 120. The stent wire of the stent body 120 may be connected to each other to prevent the hydrogel polymer 110 in the inner space from escaping to the outside.

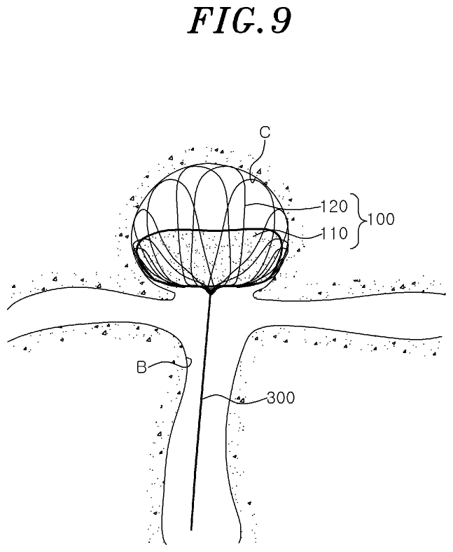

FIG. 9 is a front view illustrating a state in which a hydrogel stent for cerebral aneurysm is inserted into the cerebral aneurysm according to another modified example of the second embodiment of the present disclosure, and FIG. 10 is a side view illustrating a hydrogel stent for cerebral aneurysm according to another modified example of the second embodiment of the present disclosure.

As shown in FIGS. 9 and 10, according to another modified example of the second embodiment, the hydrogel polymer 110 having a larger volume than the inner space of the stent body 120 may be accommodated in the stent body 120. At least a portion of the hydrogel polymer 110 may be exposed in the stent body 120.

At this time, the overall length of the hydrogel polymer 110 may extend more than the height of the inner space of the stent body 120. The stent wire located in the upper side of the stent body 120 may be loosely connected to each other or partially open so that at least a portion of the hydrogel polymer 110 is exposed from the stent body 120.

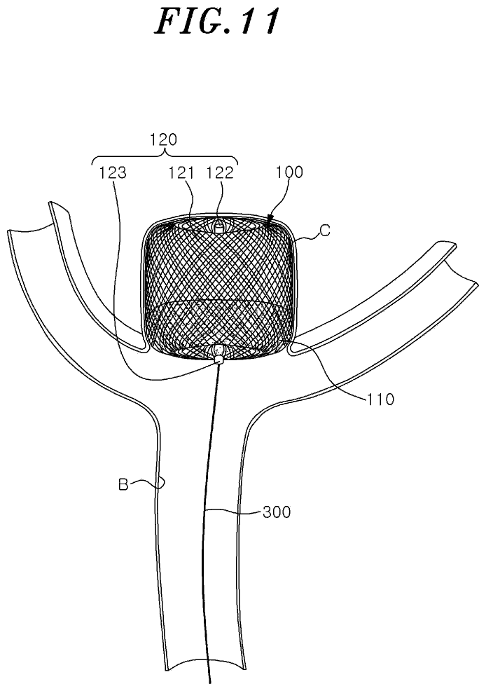

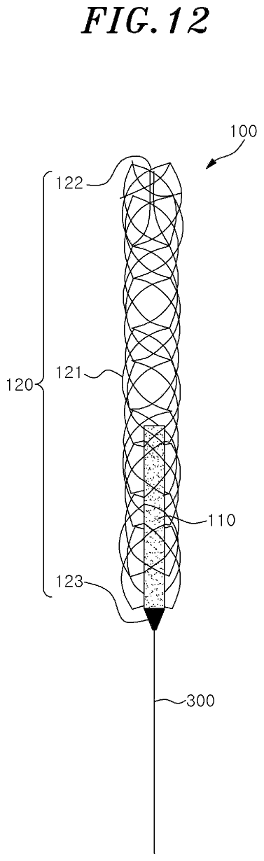

FIG. 11 is a front view illustrating a state in which a hydrogel stent for cerebral aneurysm is inserted into a cerebral aneurysm according to a third embodiment of the present disclosure, and FIG. 12 is a side view showing a hydrogel stent for cerebral aneurysm according to the third embodiment of the present disclosure.

As shown in FIGS. 11 to 12, according to the third embodiment, the stent body 120 may be provided in the form of a mesh in which the volume of the upper part is larger than the volume of the lower part. The stent body 120 may include a web portion 121, a converging portion 122, and a separation portion 123.

The web portion 121 may be connected so that a plurality of stent wires are intertwined to be convex to the outside. The web portion 121 may include an inner space in which the hydrogel polymer 110 can be accommodated. The converging portion 122 may be provided at one end of the web portion 121 so that the distal side of the web portion 121 converges.

The separation portion 123 may be provided at the other end of the web portion 121 so that the proximal side of the web portion 121 converges. The separation portion 123 may be connected to the pusher wire 300. When the stent body 120 is inserted into the cerebral aneurysm C, the separation portion 123 may be separated from the stent body 120 and then removed from the blood vessel B.

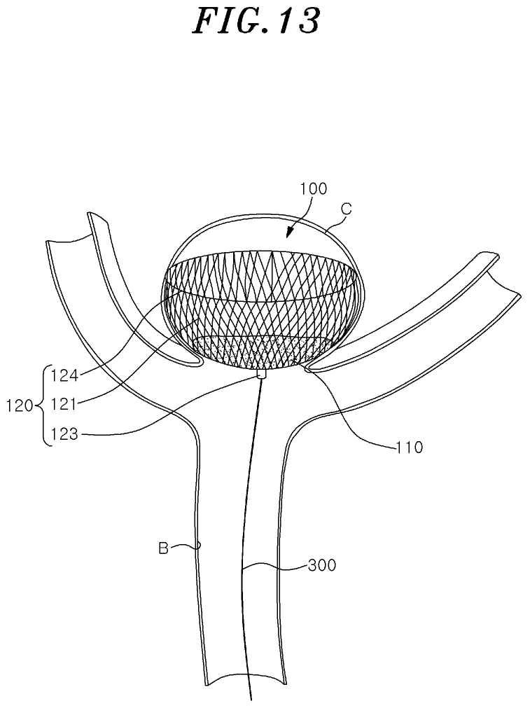

FIG. 13 is a front view illustrating a state in which a hydrogel stent for cerebral aneurysm is inserted into a cerebral aneurysm according to a modified example of the third embodiment of the present disclosure.

As shown in FIG. 13, according to the modified example of the third embodiment, the stent body 120 may be provided in the form of a mesh with an open upper end. The stent body 120 may include a web portion 121, a separation portion 123, and an opening portion 124. The web portion 121 may include an inner space in which the hydrogel polymer 110 can be accommodated.

The separation portion 123 may be provided at the other end of the web portion 121 so that the proximal side of the web portion 121 converges. The separation portion 123 may be connected to the pusher wire 300. When the stent body 120 is inserted into the cerebral aneurysm C, the separation portion 123 may be separated from the stent body 120 and then removed from the blood vessel B. The opening 124 may be provided at one end of the web 121 so that the proximal side of the web 121 is opened.

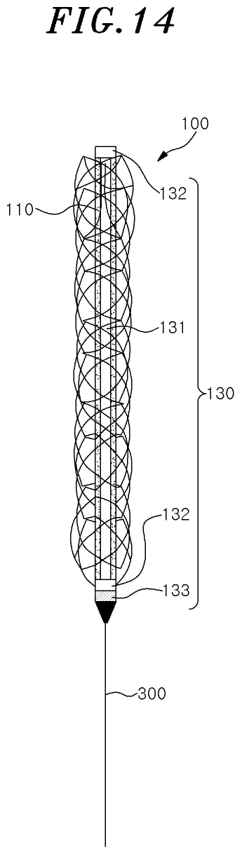

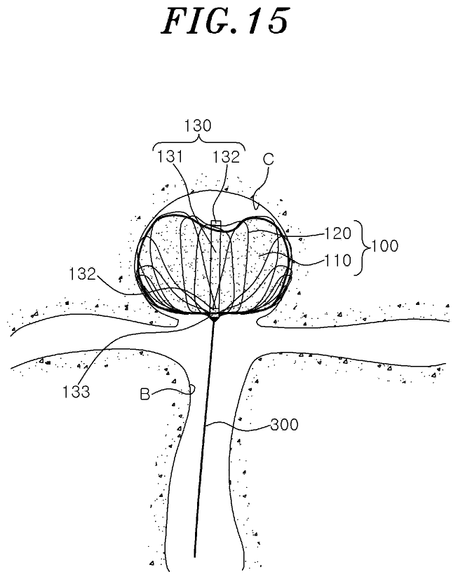

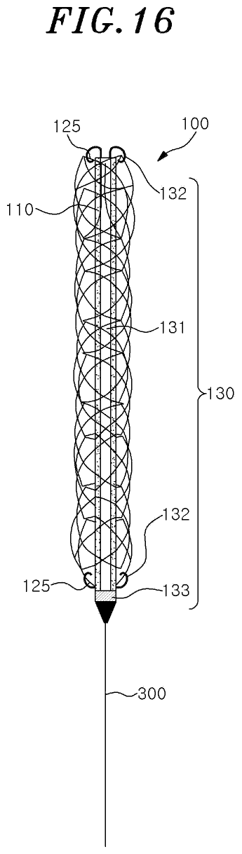

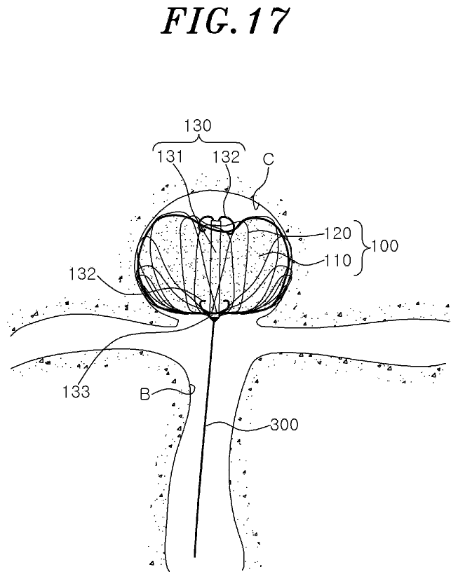

FIG. 14 shows a hydrogel stent for cerebral aneurysm according to a fourth embodiment of the present disclosure, and FIG. 15 illustrates a state in which a hydrogel stent for cerebral aneurysm is inserted into a cerebral aneurysm according to the fourth embodiment of the present disclosure. FIG. 16 shows a hydrogel stent for cerebral aneurysm which includes first coupling parts having a hook shape, and FIG. 17 illustrates a state in which the hydrogel stent for cerebral aneurysm shown in FIG. 16 is inserted into a cerebral aneurysm.

In the fourth embodiment of the present disclosure, the hydrogel stent 100 for cerebral aneurysm may include a filament 130 that can be connected to the stent body 120.

The filament 130 may be made of a polymer, a hydrogel polymer, a metal material, or combination thereof to have a predetermined rigidity to support the stent body 120. For example, the filament 130 may be manufactured by coating or attaching a hydrogel polymer to the surface of the metal material.

The filament 130 may include a filament body 131, first coupling parts 132, and a second coupling part 133.

The filament body 131 may have a predetermined length, and extend from one of longitudinal ends of the stent body 120 to the other of the longitudinal ends of the stent body 120 viewed in a longitudinal direction of the stent body so that a diameter of the unfolded hydrogel sent 100 in the longitudinal direction of the stent body 120 is maintained in the various shape of the cerebral aneurysm C.

The first coupling parts 132 may be provided at the longitudinal ends of the filament body 131 and respectively coupled to the stent body 120. The coupling between the first coupling part 132 and the stent body 120 may be performed various method. For example, the bonding by an adhesive agent may be used in the coupling between the first coupling part 132 and the stent body 120, or, as shown in FIGS. 16 and 17, the first coupling part 132 may have a hook shape and the stent body 120 may include a filament locking part 125 so that the hook shaped first coupling part 132 may be engaged with the filament locking part 125 of the stent body 120.

The second coupling part 133 may be provided at one of the longitudinal ends of the filament body 131 to be detachably attached to the pusher wire 300. The filament 130 may be detachably connected to the pusher wire 300 or fixed to the pusher wire 300. When the stent body 120 is inserted into the cerebral aneurysm C, the second coupling part 133 may be separated from the stent body 120 and then removed from the blood vessel.

Since the filament 130 is coupled to the stent body 120, a diameter in the longitudinal direction of the hydrogel stent unfolded may be maintained substantially as same as that of the hydrogel stent folded. Accordingly, it is possible to induce the stent body 120 to expand into a predetermined shape during installation within the cerebral aneurysm C, and prevent the deformation of the shape of the stent body 120 after the installation of the stent body 120. Hereinafter, the operation and effect of the cerebral aneurysm embolic device having the above-described configuration will be described.

First, referring to FIG. 1, the hydrogel stent 100 in which the hydrogel polymer 110 is accommodated in the stent body 120 may be inserted into the micro conduit 200. The hydrogel stent 100 inserted into the micro conduit 200 may be moved to the end of the micro conduit 200 along the movement path W of the micro conduit 200 by the pusher wire 300.

Referring to FIG. 2, when the micro-conduit 200 is moved to the cerebral aneurysm C along the blood vessel B and the end of the micro-conduit 200 is inserted into the cerebral aneurysm C, the stent body 120 may be unfolded while being discharged toward the cerebral aneurysm C by the push of the pusher wire 300. At this time, the stent body 120 may be expanded while being restored to its original shape.

Referring to FIG. 3, when the stent body 120 is expanded in the cerebral aneurysm C, the hydrogel polymer 110 may absorb blood within the cerebral aneurysm C and expand. Since the hydrogel polymer 110 expands in a shape corresponding to the inner surface of the cerebral aneurysm C, the hydrogel polymer 110 inflated within the cerebral aneurysm C may not be separated from the cerebral aneurysm C.

Referring to FIG. 4, when the stent body 120 is seated in the cerebral aneurysm C, the pusher wire 300 may be finally separated from the stent body 120 and removed from the blood vessel.

As described above, the present disclosure has excellent advantages such as being able to perform an embolization without detachment of the hydrogel polymer in the cerebral aneurysm by inserting the hydrogel polymer that absorbs blood and swells into the cerebral aneurysm.

The embodiments described above are merely illustrative of some examples of the technical spirit of the present disclosure, and the scope of the technical sprit of the present disclosure is not limited to the described embodiments. Those skilled in the art can change, modify, or substitute the disclosed embodiments within the scope of the present technical spirit, and it should be understood that such changes or modifications, or substitutions also belong to the scope of technical sprit of the present disclosure.

What is claimed is:

1. A hydrogel stent for cerebral aneurysm, comprising:
a hydrogel polymer; and
a stent body wrapping at least a portion of the hydrogel polymer, the stent body being expandable in a cerebral aneurysm of a blood vessel.

2. The hydrogel stent of claim 1, wherein the stent body is provided in a mesh type surrounding at least a portion of the hydrogel polymers.

3. The hydrogel stent of claim 2, wherein the hydrogel polymer is entirely accommodated in an inner space of the stent body, or a portion of the hydrogel polymer is accommodated in the stent body to be exposed outside.

4. The hydrogel stent of claim 1, wherein the stent body is formed of a Nitinol material that is restored to its original shape when its temperature is higher than a predetermined temperature or when it is in contact with water.

5. The hydrogel stent of claim 1, wherein the stent body comprises:
a web portion in which a plurality of stent wires is convexly connected to each other so as to provide an inner space in which the hydrogel polymer is accommodated;
a converging portion provided at one end of the web portion so that a distal side of the web portion converges; and
a separation portion provided at the other end of the web unit so that a proximal side of the web portion converges, the separation portion being connected to a pusher wire, and detachable when the stent body is inserted into the cerebral aneurysm.

6. The hydrogel stent of claim 1, further comprising:
a filament for supporting the stent body,
wherein the filament includes:
a filament body having a predetermined rigidity for supporting the stent body;
first coupling parts configured to be respectively coupled the stent body and provided at longitudinal ends of the filament body; and
a second coupling part configured to be coupled to a pusher wire and provided at one of the longitudinal ends of the filament body,
wherein the filament body extends from one of longitudinal ends of the stent body to the other of the longitudinal ends of the stent body viewed in a longitudinal direction of the stent body so that a diameter in the longitudinal direction of the hydrogel stent unfolded is maintained substantially as same as that of the hydrogel stent folded.

7. The hydrogel stent of claim 6, wherein the filament is formed by coating or attaching the hydrogel polymer to a surface of a metal material.

8. An embolization device for cerebral aneurysm, comprising:
a hydrogel stent for cerebral aneurysm;
a micro-conduit providing a movement path for guiding the hydrogel stent to a cerebral aneurysm of a blood vessel; and
a pusher wire connected to the hydrogel stent to be movable in the movement path;
wherein the hydrogel stent comprises:
a hydrogel polymer; and
a stent body wrapping at least a portion of the hydrogel polymer, the stent body being expandable in the cerebral aneurysm of the blood vessel.

9. The embolization device of claim 8, further comprising:
a radiopaque marker provided to the hydrogel stent and made of a material that does not pass radiation to confirm a location of the hydrogel stent in the blood vessel.

10. The embolization device of claim 8, wherein the hydrogel stent is expanded in the cerebral aneurysm while being discharged from a tip of the micro-conduit by being pushed by the pusher wire as the tip of the micro-conduit is inserted into the cerebral aneurysm.

11. The embolization device of claim 8, wherein the hydrogel stent is provided in a state of being inserted in the micro-conduit before a surgical procedure for the cerebral aneurysm, and
wherein the stent body is maintained in a state in which an outer surface thereof is compressed by an inner surface of the micro-conduit.
